(19) United States
(12) Patent Application Publication
(10) Pub. No.: US 2024/0041634 A1
(43) Pub. Date: Feb. 8, 2024

(54) An Ostomy Collection Device

(71) Applicant: Salts Healthcare Limited, Birmingham, West Midlands (GB)

(72) Inventors: Marcus Allen, Birmingham, West Midlands (GB); Mark Heynes, Stafford (GB); William Morris, Stafford (GB)

(21) Appl. No.: 18/274,534

(22) PCT Filed: Feb. 3, 2022
(86) PCT No.: PCT/GB2022/050285

§ 371 Date: Jul. 27, 2023

(30) Foreign Application Priority Data
Feb. 5, 2021 (GB) 2101650.6

(51) Int. Cl. A61F 5/44; A61F 5/445

(52) U.S. Cl. CPC A61F 5/4404; A61F 5/445

(57) ABSTRACT

A ostomy collection system and an ostomy collection device (10) including: a housing (12) formed of first and second parts, which together define a top (18), base (14) and wall (16) which enclose a collecting volume for receiving and storing waste from an ostomy appliance, the first part including the top (18) and having an inlet (30) for fluidly connecting to an outlet of an ostomy appliance so that waste from the ostomy appliance flows into the collecting volume, and the second part of the housing being disconnectable from the first part of the housing.

This invention relates to an ostomy collection device, and in particular but not exclusively to an ostomy collection device for use with a urostomy appliance.

Ostomy appliances are well known. Typically, a person having a stoma uses an ostomy appliance to collect waste from their stoma. The ostomy appliances collects a volume of waste and, once full or nearing full, the user either changes the appliance for a fresh one or empties the waste from the existing appliance.

This process works well while the user is able to visit a suitable space for changing/emptying their ostomy appliance—e.g. a bathroom or toilet. However, there may be times when the user does not wish to empty or change their appliance as often, for example, when travelling or while asleep.

The current solution is to provide the user with a flexible bag which may be supported from a structure or frame. The bag is connected to the user's ostomy appliance to increase the volume of waste that can be collected. However, the frames to which the bag is attached can be unstable and are easily knocked over.

Embodiments of the present invention seek to address one or more problems associated with the prior art.

Various aspects of the invention are provided in the appended claims.

Embodiments of the present invention are described, by way of example only, with reference to the accompanying drawings, in which:

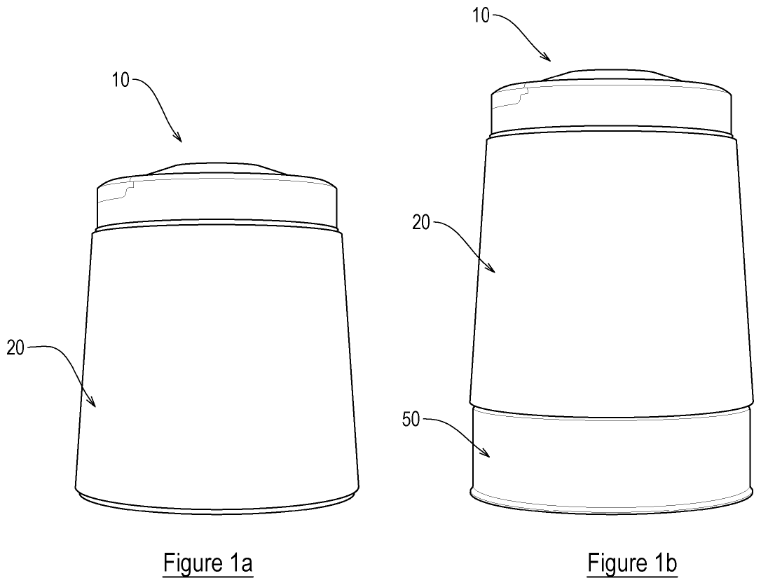

FIGS. 1a and 1 b illustrate an ostomy collection device in accordance with an embodiment of the present invention,

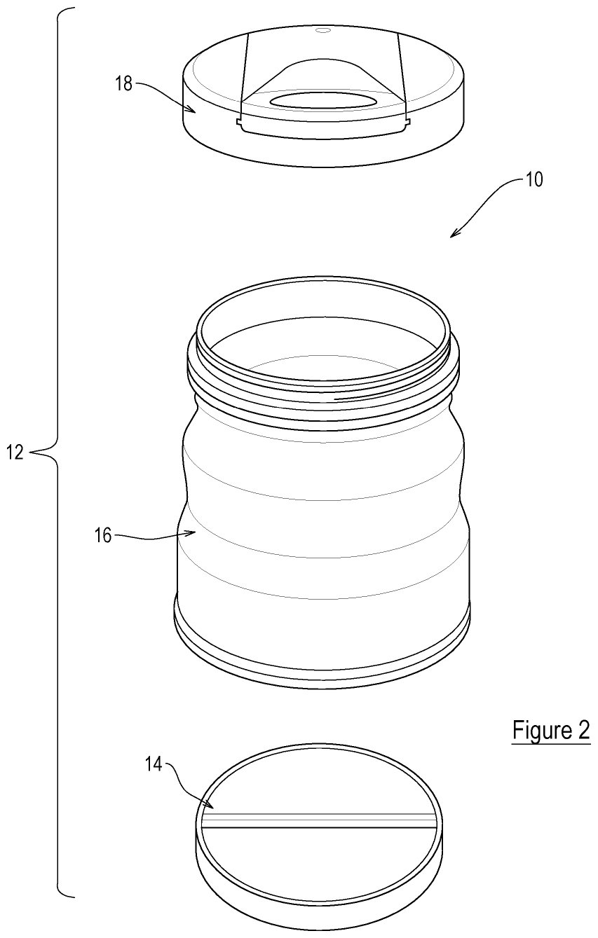

FIG. 2 is an exploded view of the ostomy collection device,

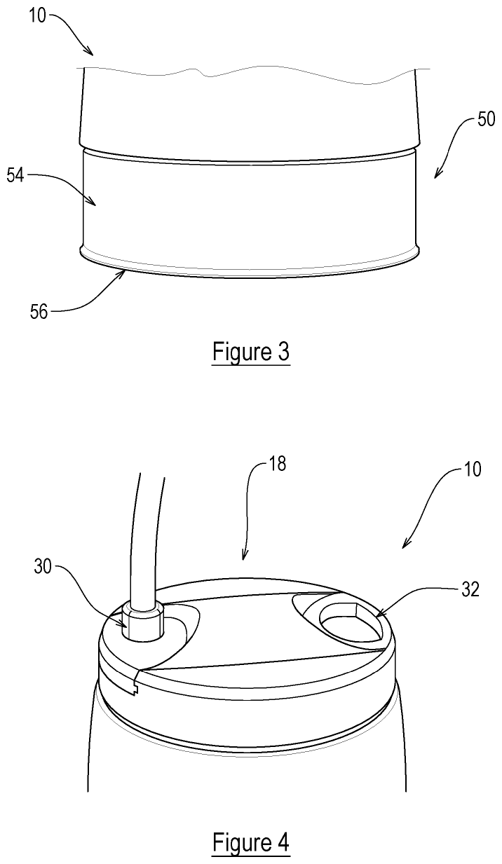

FIG. 3 illustrates a part of the ostomy collection device,

FIG. 4 is a perspective view of a part of the ostomy collection device,

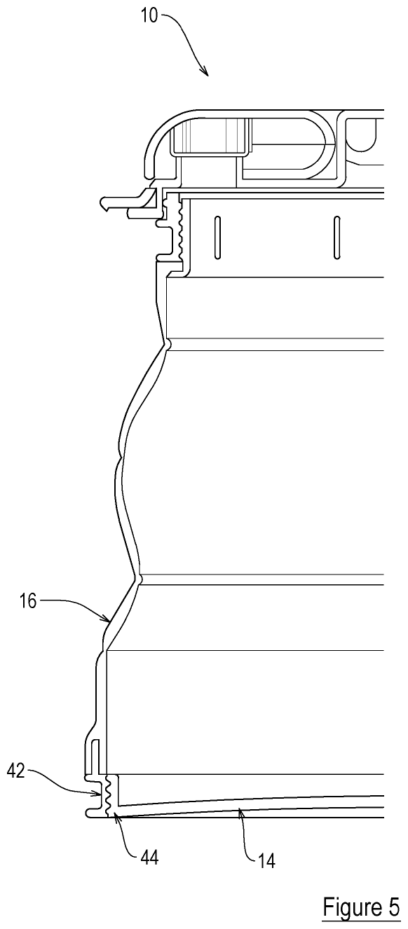

FIG. 5 is a cross-sectional view of a part of the ostomy collection device,

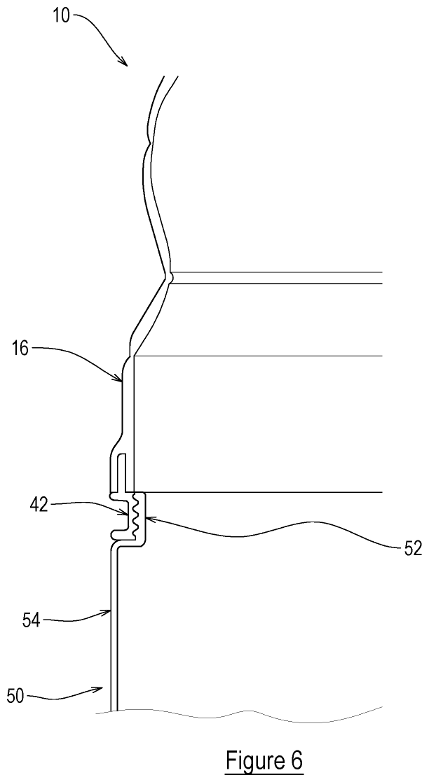

FIG. 6 is a cross-sectional view of a part of the ostomy collection device.

An ostomy collection device 10 is shown in FIGS. 1a and 1b. The ostomy collection device 10 is configured to connect to an outlet of an ostomy appliance. In use, the ostomy appliance connects about a user's stoma (and collects waste exiting the stoma).

The ostomy collection device 10 includes a housing 12 (shown in FIG. 2). The housing 12 is formed of a first part and a second part, which together define a collecting volume for receiving and storing waste from an ostomy appliance. The first and second parts of the housing are disconnectable from one another. This allows the first part of the housing 12 to be connected to a third housing part 50 (discussed later) to alter the internal volume of the housing 12.

In the illustrated example, the first part of the housing 12 is provided by a wall 16 and a top 18 and the second part of the housing is provided by a base 14. When the first and second parts are connected together, the wall 16 extends between the base 14 and the top 18 to enclose the collecting volume.

The housing 12 further includes an inlet 30 (shown in FIG. 4). The inlet 30 is configured to connect to an outlet of an ostomy appliance (not shown), so that waste from the ostomy appliance flows into the collecting volume. The inlet 32 is positioned on the top 18 of the housing. In some embodiments, the housing 12 includes an outlet 32, which allows the contents of the collecting volume to be disposed of (e.g. into a toilet, etc.). In some embodiments, the outlet 32 is positioned on top 18 of the housing 12.

The first part includes a first engaging formation 42 (in the present example, the first engaging formation 42 is formed on or as a part of the wall 16). The second part includes a second engaging formation 44 (shown in FIG. 5). In the illustrated example, the second engaging formation 44 is formed on or as part of the base 14. Engagement of the two respective formations 42, 44 connects the first and second parts of the housing 12 together.

In some embodiments, the first engagement formation 42 and second engagement formation 44 are corresponding helical threaded portions. Thus, on relative rotation of the two helical threads together, the base 14 and the wall 16 (in other words, the first part and the second part) are connected together or disconnected from each other depending on the relative direction of rotation. It should be appreciated that the first engaging formation 42 and second engaging formation 44 could include other mechanisms for connecting and disconnecting the base 14 from the wall 16 such as a protrusion/recess, magnets or other mechanisms which allow the base 14 and wall 16 to be connected and disconnected.

The base 14 and top 18 are (when viewed from directly above the housing 12) substantially circular (although it should be appreciated that other shapes are also possible and the shapes of the base 14 and the top 18 do not have to match necessarily). In embodiments, there is a single continuous wall 16, which extends around the entire periphery of the top 18 (and base 14 when the two housing parts are connected). In the illustrated examples, the wall 16 is substantially cylindrical in shape, e.g. it is ring-shaped or annular.

In some embodiments, the wall 16 is ridged or concertina-shaped (shown in FIG. 2). The ridged or concertinaed wall 16 of the ostomy collection device 10 may be manipulated so that at least a portion of the wall 16 is folded upon itself or unfolded from a folded state.

The third part 50 of the housing is configured to be connectable to and disconnectable from the first part. In some embodiments, the third part 50 includes a base 54 and a wall 56. The wall 56 is connected to the base 54 to form an open topped container. It should be appreciated that the second part of the housing 12 could have similar form to the third part 50 (this would increase the collecting volume enclosed relative to the second part being the base 14 only).

In embodiments, the width of the third part 50 remains substantially constant between the base 54 and the top of the wall 56 (although this does not need to be the case and the base 54 of the third part 50 may be wider than the top of the wall 56 or vice versa). The width of the top of the wall 56 of the third part 50 is of a similar or identical width to the bottom of the first part.

In embodiments, the profile of the base 54 when viewed from directly above the third part 50 is substantially circular. In some embodiments, the wall 56 extends continuously around the periphery of the base 54 to form a ring-shaped or annular wall. However, it should be appreciated that the third part 50 does not necessarily have to be circular.

In the present example, the third part 50 of the housing has the same or similar outer profile as the first part of the housing 12, which makes it easier to connect the two together as is described below.

In embodiments, the third part 50 includes a third engaging formation 52. The third engaging formation 52 is configured to engage with the first engaging formation 42 of the first part of the housing 12 (shown in FIG. 6).

In some embodiments, the third engaging formation 52 is formed on or as part of the wall 56. The third engaging formation 52 is a corresponding helical thread which is configured to cooperate with the first engaging formation 42. On relative rotation of the two threads together, the third part 50 and the first part of the housing 12 are connected together or disconnected from each other depending on the relative direction of rotation.

As described above in relation to the first engaging formation 42, the third engaging formation 52 could include other mechanisms for connecting and disconnecting third part 50 to/from the first part, for example, a protrusion/recess, magnets or other mechanism.

The system provided by the three housing parts provides a reconfigurable ostomy collection device 10, which is able to provide two different volumes for the collecting volume depending on whether the first part and the second parts are connected together or the first and the third parts are connected together.

The first state/volume is provided by the first and second parts of the housing 12 (shown in FIG. 1a and FIG. 5). In this first state, the ostomy collection device 10 has a first collecting volume. The second state/volume is provided by the first and third parts of the housing 12 (shown in FIG. 1b and FIG. 6). In this second state, the ostomy collection device 10 has a second collecting volume. The second and third parts could be identical to one another but preferably they will be different sizes so that when each is connected to the first part, they provide a different sized collecting volume.

It should be appreciated that additional parts of the housing 12 of different sizes could be provided to increase the range of volumes available to a user. An ostomy collection device 10 that is reconfigurable between two states is advantageous as it enables the user to alter the collecting volume of the ostomy collection device 10 as desired. Furthermore, the user can preconfigure the ostomy collection device 10 into one of the two states dependent on the duration in which they estimate they will not be able to empty the contents of the collecting volume.

The following clauses outline optional features and combinations of features.

1. An ostomy collection device including:
a housing formed of first and second parts, which together define a top, base and wall which enclose a collecting volume for receiving and storing waste from an ostomy appliance,
the first part including the top and having an inlet for fluidly connecting to an outlet of an ostomy appliance so that waste from the ostomy appliance flows into the collecting volume, and
the second part of the housing being disconnectable from the first part of the housing.

2. An ostomy collection device according to clause 1 wherein the first part of the housing includes a first engaging formation and the second part includes a second engaging formation.

3. An ostomy collection device according to clause 2 wherein the first and second engaging formations are corresponding helical threaded portions.

4. An ostomy collection device according to clause 2 or 3 wherein the first part of the housing includes at least a portion of the wall and the first engagement portion is formed on/as part of the wall.

5. An ostomy collection device according to any preceding clause wherein the second part of the housing includes the base.

6. An ostomy collection device according to any preceding clause further including a third housing part which is configured to be connectable to and disconnectable from the first part of the housing.

7. An ostomy collection device according to clause 6 wherein the third housing part includes:
a base, and
a wall extending from the base, and
such that, when the third housing part is connected to the first housing part it provides the base of the housing.

8. An ostomy collection device according to clause 7 wherein the third housing part includes a third engaging formation, and wherein the first and third engaging formations are corresponding helical threaded portions.

9. An ostomy collection device according to clause 8 wherein the third engaging formation is formed on/part of the wall of the third housing part.

10. An ostomy collection device according to any preceding clause wherein the top of the first part of the housing is disconnectable from the wall of the first part of the housing.

11. An ostomy collection device according to any preceding clause further including an outlet which is separate from the inlet for waste to be emptied from the collecting volume, and wherein the outlet is located on the top of the first part of the housing.

12. An ostomy collection device including:
a housing formed of first and second parts, which enclose a collecting volume for receiving and storing waste from an ostomy appliance,
the first and second parts of the housing being disconnectable from each other, so that the first part of the housing is connectable to a third housing part to form an alternative collecting volume.

13. An ostomy collection device according to clause 12 wherein the first, second and third parts of the housing include respective first, second and third engaging formations.

14. An ostomy collection device according to clause 13 wherein the first engaging formation is configured to engage with the second and third engaging formations.

15. An ostomy collection device according to clause 13 or 14 wherein the first, second and third engaging formations are helical threads.

16. An ostomy collection device according to any one of clause 12 to 15 wherein the first part includes the top, the top having an inlet for fluidly connecting to an outlet of an ostomy appliance so that waste from the ostomy appliance flows into the collecting volume.

17. An ostomy collection device according to any one of clauses 12 to 16 which includes any one or more features outlined in any one of clauses 1 to 11 or clauses 18 to 23.

18. An ostomy collection system for providing a reconfigurable ostomy collection device, the system including:
a first part, a second part and a third part,
the first and second parts being connectable so as to form a first collecting volume, and
the first and third parts being connectable so as to form a second collecting volume, wherein the first part is configured such that it is connectable to either the second or third part.

19. An ostomy collection system according to clause 18 wherein the first and second collecting volumes are different sizes.

20. An ostomy collection system according to clause 18 or 19 wherein the first part of the housing includes a first engaging formation and the second and third parts include corresponding engaging formations, wherein optionally the first engaging portion and the corresponding engaging formations each include a threaded portion.

21. An ostomy collection system according to any of clauses 18 to 20 wherein the first part includes a top and a wall, and preferably wherein the top has an inlet for fluidly connecting to an outlet of an ostomy appliance so that waste from the ostomy appliance flows into the first/second collecting volume

22. An ostomy collection system according to clause 21 wherein the top of the first part is disconnectable from the wall of the first part of the housing.

23. An ostomy collection system according to clause 21 or 22 wherein the first part includes an outlet, which is separate from the inlet for waste to be emptied from the collecting volume, and optionally wherein the outlet is located on the top of the first part of the housing.

24. An ostomy collection system according to any one of clauses 18 to 23 wherein the second and third parts each provides a base for supporting the ostomy collection device when in the first state or the second state, respectively.

25. An ostomy collection device according to any one of clauses 18 to 24 which includes any one or more features outlined in any one of clauses 1 to 16.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

Although certain example embodiments of the invention have been described, the scope of the appended claims is not intended to be limited solely to these embodiments. The claims are to be construed literally, purposively, and/or to encompass equivalents.

1. An ostomy collection device including:
a housing formed of first and second parts, which together define a top, base and wall which enclose a collecting volume for receiving and storing waste from an ostomy appliance,
the first part including the top and having an inlet for fluidly connecting to an outlet of an ostomy appliance so that waste from the ostomy appliance flows into the collecting volume, and
the second part of the housing being disconnectable from the first part of the housing.

2. An ostomy collection device according to claim 1 wherein the first part of the housing includes a first engaging formation and the second part includes a second engaging formation.

3. An ostomy collection device according to claim 2 wherein the first and second engaging formations are corresponding helical threaded portions.

4. An ostomy collection device according to claim 2 wherein the first part of the housing includes at least a portion of the wall and the first engagement portion is formed on/as part of the wall.

5. An ostomy collection device according to claim 1 wherein the second part of the housing includes the base.

6. An ostomy collection device according to claim 1 further including a third housing part which is configured to be connectable to and disconnectable from the first part of the housing.

7. An ostomy collection device according to claim 6 wherein the third housing part includes:
a base, and
a wall extending from the base, and
such that, when the third housing part is connected to the first housing part it provides the base of the housing.

8. An ostomy collection device according to claim 7 wherein the third housing part includes a third engaging formation, and wherein the first and third engaging formations are corresponding helical threaded portions.

9. An ostomy collection device according to claim 8 wherein the third engaging formation is formed on/part of the wall of the third housing part.

10. An ostomy collection device according to claim 1 wherein the top of the first part of the housing is disconnectable from the wall of the first part of the housing.

11. An ostomy collection device according to claim 1 further including an outlet which is separate from the inlet for waste to be emptied from the collecting volume, and wherein the outlet is located on the top of the first part of the housing.

12. An ostomy collection device including:
a housing formed of first and second parts, which enclose a collecting volume for receiving and storing waste from an ostomy appliance,
the first and second parts of the housing being disconnectable from each other, so that the first part of the housing is connectable to a third housing part to form an alternative collecting volume.

13. An ostomy collection device according to claim 12 wherein the first, second and third parts of the housing include respective first, second and third engaging formations.

14. An ostomy collection device according to claim 13 wherein the first engaging formation is configured to engage with the second and third engaging formations.

15. An ostomy collection device according to claim 13 wherein the first, second and third engaging formations are helical threads.

16. An ostomy collection device according to claim 12 wherein the first part includes the top, the top having an inlet for fluidly connecting to an outlet of an ostomy appliance so that waste from the ostomy appliance flows into the collecting volume.

17. An ostomy collection system for providing a reconfigurable ostomy collection device, the system including:
a first part, a second part and a third part,
the first and second parts being connectable so as to form a first collecting volume, and
the first and third parts being connectable so as to form a second collecting volume, wherein the first part is configured such that it is connectable to either the second or third part.

18. An ostomy collection system according to claim 17 wherein the first and second collecting volumes are different sizes.

19. An ostomy collection system according to claim 17 wherein the first part of the housing includes a first engaging formation and the second and third parts include corresponding engaging formations, wherein optionally the first engaging portion and the corresponding engaging formations each include a threaded portion.

20. An ostomy collection system according to claim 17 wherein the first part includes a top and a wall, and preferably wherein the top has an inlet for fluidly connecting to an outlet of an ostomy appliance so that waste from the ostomy appliance flows into the first/second collecting volume

21. An ostomy collection system according to claim 20 wherein the top of the first part is disconnectable from the wall of the first part of the housing.

22. An ostomy collection system according to claim 20 wherein the first part includes an outlet, which is separate from the inlet for waste to be emptied from the collecting volume, and optionally wherein the outlet is located on the top of the first part of the housing.

23. An ostomy collection system according claim 17 wherein the second and third parts each provides a base for supporting the ostomy collection device when in the first state or the second state, respectively.
